(19) United States
(12) Patent Application Publication
(10) Pub. No.: US 2024/0023568 A1
(43) Pub. Date: Jan. 25, 2024

(54) FAT ENCAPSULATED MICROBIAL CULTURES

(71) Applicant: Chr. Hansen A/S, Hoersholm (DK)

(72) Inventors: Surender Kumar Dhayal, Hoersholm (DK); Anisha Goel, Hoersholm (DK); Simranjeet Tiwana, Hoersholm (DK)

(73) Assignee: Chr. Hansen A/S, Hoersholm (DK)

(21) Appl. No.: 18/254,711

(22) PCT Filed: Nov. 29, 2021
(86) PCT No.: PCT/EP2021/083376

§ 371 Date: May 26, 2023

(30) Foreign Application Priority Data
Nov. 30, 2020 (EP) 20210730.6

(51) Int. Cl. A23C 9/123; A23L 33/135; A23P 10/35; C12N 1/20; C12N 1/04

(52) U.S. Cl. CPC A23C 9/1232; A23L 33/135; A23P 10/35; C12N 1/20; C12N 1/04; C12R 2001/225

(57) ABSTRACT

The present invention relates to encapsulation of microbial cultures to improve the robustness and stability upon storage. In particular, the present invention relates to dry preparations of microbial cultures, such as lactic acid bacteria (LAB), coated by a fat-matrix that increase survivability and mitigate post-acidification upon storage at ambient temperature for extended periods of time.

### TECHNICAL FIELD OF THE INVENTION

The present invention relates to encapsulation of microbial cultures to improve the robustness and stability upon storage. In particular, the present invention relates to dry preparations of microbial cultures, such as lactic acid bacteria (LAB), coated by a fat-matrix that increase survivability and mitigate post-acidification upon storage at ambient temperature for extended periods of time.

### BACKGROUND OF THE INVENTION

An important characteristic for most goods intended for consumption is the stability upon storage. Goods with a short shelf life or demanding storage conditions are difficult to handle and thus not as enticing from a commercial or consumer point of view as more robust goods. The single most important factor influencing the expected shelf life and safety of products for consumption is the presence of unwanted microorganisms. Refrigerated storage is therefore a requirement for many products to prevent fouling and associated health risks.

In contrast to unwanted microorganisms, microbial cultures, such as lactic acid bacteria (LAB), may also serve a beneficial purpose in fermented dairy foods and beverages as they can help to improve the nutritional and organoleptic characteristics, as well as extend the shelf life. Some strains of LAB have been reported to exhibit health benefits to human and animals and may thereby be referred to as probiotic strains. These probiotics are typically provided separately as powdered compositions, e.g. as freeze dried (FD) powders, and mixed with additional ingredients to yield a final product. Addition of the microbial culture is normally completed after pasteurization to retain viability of the beneficial microbial culture.

Pasteurization is intended to destroy or deactivate organisms and enzymes that contribute to spoilage or risk of disease. However, since pasteurization is not sterilisation it does not kill bacterial spores, which is why refrigeration is necessary to avoid germination of unwanted microorganisms. However, refrigeration is very expensive, energy consuming and in some cases not feasible in e.g. developing countries or remote regions where cold transport is not possible. Moreover, the final product may be an article that is not readily stored under refrigerated conditions.

To avoid refrigeration, a second step of pasteurization may be performed to extend the expected shelf life of products for consumption by rendering inert any microorganisms that may have germinated in the product from the stage of initial pasteurization until finalisation of the product. Many products for consumption, such as dairy products, may therefore extend their shelf life by undergoing a second step of pasteurization at the end of acidification (fermentation). These products are termed post-pasteurised products and include e.g. post-pasteurised yoghurt (PPY). Unfortunately, not many microbial cultures, such as lactic acid bacteria, can survive the post-pasteurization step making it quite challenging to deliver viable probiotics in sufficient count and without significant loss of cell count by addition of the probiotics along with starter culture.

Viable probiotics in post-pasteurised products is desired from a health perspective and a technological solution to achieve this is to add the probiotics by sterile in-line mixing after the post-pasteurization step. However, this technological solution requires a significant investment involving modifications in the existing production lines, making it unfeasible in many instances.

Another challenge with maintaining viability of probiotics relates to post-acidification of the dairy product. Starter cultures tend to produce lactic acid from lactose during storage. This happens even at refrigerated temperatures and causes the phenomenon known as post-acidification. The same is the case for probiotic bacteria, although at a lower rate. The acidic environment has a negative effect on the viability of the probiotics and moreover impairs the consumer experience.

It would therefore be advantageous with a solution enabling the utility of microbial cultures in post-pasteurised products by introduction directly into existing production lines. Specifically, the provision of a microbial culture capable of (i) maintaining viability upon the post-pasteurization step and (ii) not showing significant post-acidification during shelf storage under ambient conditions.

### SUMMARY OF THE INVENTION

The present invention relates to encapsulation of microbial cultures in a fat matrix. In particular, the present invention discloses methods for preparing dry microbial cultures with a protective coating of fat which allows the microbial culture to maintain viability during a post-pasteurization step and throughout subsequent storage at ambient temperature. The fat encapsulated microbial cultures constitute an improved biocompatible option for applications wherein the microbial culture must be added prior to a pasteurization step.

Accordingly, an object of the present invention relates to the provision of methods for preparing microbial cultures that may be used directly in existing production lines wherein post-pasteurization is desired.

In particular, it is an object of the present invention to provide an improved dry microbial culture that retain cell viability upon post-pasteurization and storage at ambient temperature.

Thus, an aspect of the present invention relates to a fat encapsulated microbial culture comprising:
i) a preparation comprising a microbial culture, and
ii) an encapsulation matrix comprising one or more fat components, each of which fat components having a melting point of at least 25° C.

Another aspect of the present invention relates to a composition comprising the fat encapsulated microbial culture as described herein.

Yet another aspect of the present invention relates to a dairy product comprising a fat encapsulated microbial culture or a composition as described herein.

Still another aspect of the present invention relates to a method for preparing a fat encapsulated microbial culture or a composition as described herein, said method comprising the steps of:
i) provision of a preparation comprising a microbial culture,
ii) provision of an encapsulation matrix, and
iii) mixing the encapsulation matrix with the preparation to form a microencapsulated microbial culture,

wherein the encapsulation matrix comprises one or more fat components, each of which fat components having a melting point of at least 25° C.

A further aspect of the present invention relates to a fat encapsulated microbial culture or a composition as described herein obtainable by a method as described herein.

A still further aspect of the present invention relates to use of a fat encapsulated microbial culture or a composition as described herein in a product selected from the group consisting of a feed, a plant health product, a food, a beverage and/or a pharmaceutical product.

An even further aspect of the present invention relates to a method for preparing a post-pasteurized yoghurt (PPY), said method comprising the steps of:
i) provision of a yoghurt with a pH in the range of 4.0-4.6,
ii) addition of a fat encapsulated microbial culture or a composition as described herein to the yoghurt, and
iii) post-pasteurization of the yoghurt of step ii).

### BRIEF DESCRIPTION OF THE FIGURES

FIG. 1 shows the Log₁₀ loss in cell viability (CFU/g) of both ‘Free’ and ‘Microencapsulated’ (ME) freeze-dried LGG in a post pasteurized yogurt model at pH 4.5 during 56 days storage at 5° C. or 25° C.

FIG. 2 shows pH measurement of both ‘Free’ and ‘Microencapsulated’ (ME) freeze-dried LGG in a post pasteurized yogurt model at pH 4.5 during 56 days storage at 5° C. or 25° C.

The present invention will in the following be described in more detail.

### DETAILED DESCRIPTION OF THE INVENTION

### Definitions

Prior to outlining the present invention in more details, a set of terms and conventions is first defined:

Microbial Culture

In the present context, the term “microbial culture” refers to a population of microorganisms. Microorganisms include all unicellular organisms, such as archaea and bacteria, but also many multicellular organisms, such as fungi and algae. Microbial cultures as referred to herein does not include unwanted microorganisms that contribute to spoilage or risk of disease.

Probiotic Culture

In the present context, the terms “probiotic” or “probiotic culture” refers to microbial cultures which, when ingested in the form of viable cells by humans or animals, confer an improved health condition, e.g. by suppressing harmful microorganisms in the gastrointestinal tract, by enhancing the immune system or by contributing to the digestion of nutrients. Probiotics may also be administered to plants. Probiotic cultures may comprise bacteria and/or fungi.

Lactic Acid Bacteria (LAB)

In the present context, the term “lactic acid bacteria (LAB)” refers to a group of Gram positive, catalase negative, non-motile, microaerophilic or anaerobic bacteria that ferment sugar with the production of acids including lactic acid as the predominantly produced acid, acetic acid, formic acid and propionic acid. The industrially most useful lactic acid bacteria include, but are not limited to, Lactobacillus species (spp.), Lactococcus spp., Streptococcus spp., Leuconostoc spp., Pediococcus spp., Brevibacterium spp, Enterococcus spp. and Propionibacterium spp. Additionally, lactic acid producing bacteria belonging to the group of the strict anaerobic bacteria, Bifidobacteria, i.e. Bifidobacterium spp. which are frequently used as food starter cultures alone or in combination with lactic acid bacteria, are generally included in the group of lactic acid bacteria. Even certain bacteria of the genus Staphylococcus (e.g. S. carnosus, S. equorum, S. sciuri, S. vitulinus and S. xylosus) have been referred to as LAB (Seifert & Mogensen (2002)).

Fat Encapsulated

In the present context, the term “fat encapsulated” refers to an entity containing a coating or layer, which secludes it from the surrounding environment. Thus, a fat encapsulated microbial culture is a microbial culture which are compartmentalized into distinct entities separated from each other and the medium into which they are dispersed.

Encapsulation Matrix

In the present context, the term “encapsulation matrix” refers to the coating or layer enclosing the microbial culture. The coating or layer comprises at least one fat component, but may also comprise components selected from other categories, such as emulsifiers. Preferably, all components of the encapsulation matrix are food graded, such as Generally Recognized as Safe (GRAS) ingredients.

Fat Component

In the present context, the term “fat component” refers to a compound or mixture of compounds that is soluble in non-polar solvents and is in a solid or semi-solid state at room temperature. The fat components may be mono-, di- and triglycerides, phospholipids, sterols, waxes or free fatty acids, or a mixture thereof. Specifically, glycerides are esters formed from glycerol and fatty acids. Preferably, the fat component comprises a high amount of fatty acids with melting points above room temperature.

Importantly, the fat components referred to herein are distinct from most oils, which are fluid at room temperature.

Fatty Acid

In the present context, the term “fatty acid” refers to carboxylic acids comprising a long aliphatic carbon chain. The chain may be of varying length and is either saturated of unsaturated. Fatty acids may be saturated at different degrees, meaning that they comprise in order of decreasing saturation e.g. 1, 2, 3, 4, 5 or 6 double bonds. In general terms, longer chain lengths and increased degree of saturation elevates the melting point of fatty acids and their derivatives.

Preferred fat components therefore comprise fatty acids with a high degree of saturation and/or long carbon chain length. These include, but are not limited to, capric acid, lauric acid, myristic acid, palmitic acid, stearic acid, arachidic acid, behenic acid, lignoceric acid, cerotic acid, elaidic acid, vaccenic acid, erucic acid and hydroxystearic acid.

Interesterified Oils

In the present context, the term “interesterified oils” refers to a fat component wherein the fatty acids have been rearranged by breaking and reforming the ester bonds that link the fatty acid to glycerol. This is typically done by use of a catalyst. Interesterification may be used to elevate the melting point of liquid oils by combining them with solid fats.

The term “interesterified oil” may be used interchangeable with the term “interesterified fat”.

Melting Point

In the present context, the term “melting point” refers to the temperature at which a substance, such as a fat component, changes its state from solid to liquid. Herein the melting point is specified at standard pressure of 1 atm.

The melting point as referred to herein is the slip melting point. The slip melting point of the fat components can be measured by the standard method as described in AOCS Cc 3-25.

Emulsifier

In the present context, the term “emulsifier” refers to a substance that stabilizes an emulsion by helping two liquids to mix. Emulsifiers are compounds that typically have a polar or hydrophilic part and a non-polar or hydrophobic part. Examples of emulsifiers include, but are not limited to, monoglycerides, diglycerides, triglycerides, lecithin, milk proteins, gums and combinations thereof.

Viability

In the present context, the term “viability” refers to living cells in a culture. Thus, the viability of a cell culture may be determined by measuring the number of colony forming units (CFU). CFU refer to the number of individual colonies of any microbe that grow on a plate of media. This value in turn represents the number of bacteria or fungi capable of replicating as they have formed colonies on the plate.

In brief, the CFU/g can be determined as follows; A known amount of sample (e.g. freeze dried) is homogenized with a specific volume of diluent (1:100), using a stomacher, the solution is then resuspended by using a vortex mixer and is then subjected to decimal dilutions in peptone saline diluent (also referred to as ‘maximum recovery diluent (MRD)’). MRD comprises peptone, NaCl and demineralised water. Dilutions are poured on the plates, mixed with MRS Agar (Hi-media, M641) and incubated at 37° C. until visible colony growth. After incubation, colonies are counted manually. For fat-coated samples, the samples are typically incubated at 40° C. for 30 min in a diluent comprising MRD with Polysorbate 80 (may also be termed Tween 80) to ensure complete release of cells from the matrix.

Water Activity

In the present context, the term “water activity” refers to the partial vapour pressure of water in a substance divided by the standard state partial vapour pressure of water. The water activity is denoted Aw. Specifically, Aw of a food is the ratio between the vapor pressure of the fat encapsulated microbial culture itself, when in a completely undisturbed balance with the surrounding air media, and the vapor pressure of distilled water under identical conditions.

As a general note, water migrates from areas of high Aw to areas of low Aw. The storage stability of food products can typically be extended by formulating the product with low Aw.

Water activity is measured using a Rotronics water activity analyzer with HC2-AW probe. This probe is fitted with a HYGROMER® WA-1/Pt-100, 1/3 DIN Class B humidity sensor and the psychrometric calculations are done by the dew or frost point method.

Briefly, sample is placed into a sample cup and filled up to within 3 mm of the rim while ensuring as less air in the container as possible to ensure a faster equilibration time. Next, the measurement head is placed on the sample holder ensuring a tight seal. The water activity is the measured using a predictive model of the Rotronic water activity analyser.

The water activity is calculated using the following formulae: Aw=p/pₛ and %ERH=100×Aw, where, p=partial pressure of water vapor at the surface of the product; pₛ=saturation vapor/partial pressure of water vapor above pure water at the product temperature; ERH=Equilibrium Relative Humidity.

Storage Stability

In the present context, the term “storage stability” refers to the ability of a fat encapsulated microbial culture to maintain viability when part of a product over an extended duration of time at a temperature of 25° C. for a period of at least 2 weeks, such as at least 4 weeks, preferably at least 6 weeks, more preferably at least 8 weeks.

Storage stability can be determined by analysing how the count of viable microbial cells develop over time. Viability of the microbial culture is measured by determining the CFU/g as described herein. Thus, a measure of the storage stability of the fat encapsulated microbial culture in a product may be determined by evaluating CFU/g of the fat encapsulated microbial culture in a product at time point 0 (just after post-pasteurization) and at any point of subsequent storage at accelerated storage conditions, such as after 2 weeks, 4 weeks, 6 weeks or 8 weeks.

Hydrophobic Coating

In the present context, the term “hydrophobic coating” refers to a hydrophobic layer or shell that is positioned on the surface of fat encapsulated microbial culture. Such hydrophobic layer or shell may comprise one or more hydrophobic compounds or molecules comprising a hydrophobic moiety that increases hydrophobicity of the outer surface of the fat encapsulated microbial culture.

Feed

In the present context, the term “feed” refers to a food given to domestic animals. Domesticated animals include, but are not limited to, pets, such as dogs, cats, rabbits, hamsters and the like, livestock, such as cattle, sheep, pigs, goats and the like, and beast of burden, such as horses, camels, donkeys and the like.

Feed may be blended from various raw materials and additives and specifically formulated according to the requirements of the recipient animal. Feed may be provided e.g. in the form of mash feed, crumbled feed or pellet feed.

The term “feed” includes also premixes, which are composed of ingredients such as vitamins, minerals, chemical preservatives, antibiotics, fermentation products and combinations thereof. Premixes are typically added as a nutritional supplement to the feed given to the domestic animals.

Post Pasteurised Product

In the present context, the term “Post pasteurised product” refers to a product, which has undergone two pasteurisation steps to enhance the shelf life of the product. Typically, the first step of pasteurisation is performed in the initial processing of the product and the second step is performed as the last processing step.

Post pasteurised product include both final products and additives intended to be mixed with other ingredients to form a final product.

Post Pasteurised Yoghurt (PPY)

In the present context, the term “Post pasteurised yoghurt (PPY)” refers to a class of yoghurts which undergo an initial pasteurisation step at the start of processing and a second step of pasteurisation at the end of acidification (fermentation).

PPY is characterised by their extended shelf life under non-refrigerated conditions, such as at 25° C. Therefore, PPY may also be referred to as ambient stable yoghurt.

Fat Encapsulated Microbial Cultures

It is highly desirable to deliver beneficial microbial cultures to consumers via ingestible products, such as a variety of dairy products. Microbial cultures may be probiotic in nature and therefore add nutritional value and aid the health of the consumer. Such microbial cultures are typically acquired separately as powdered compositions and mixed with additional ingredients to yield a final product. However, for post-pasteurised products in which a second pasteurisation step is applied to increase storage stability at ambient temperature, it is problem that the secondary heat treatment significantly reduces the amount of viable beneficial microbes. Moreover, it is a challenge to maintain a viable pool of microbial cultures in dairy products upon storage because of post-acidification negatively affecting the viability.

To effectively provide post-pasteurised products, such as dairy products, with viable microbial cultures to consumers it is necessary to devise new formulations and methods that enable their utilization directly in existing production lines. Thus, the microbial cultures should be capable of surviving the secondary pasteurisation step and not display significant post-acidification upon subsequent storage at ambient temperatures.

Herein are set out methods for encapsulating microbial cultures in a matrix comprising one or more fat components. Without being bound theory, the encapsulation matrix assists in absorbing heat from the environment and protect the cells during the time scale of heating. Moreover, it was found that the fat encapsulation efficiently mitigates any significant post-acidification of the microbial culture upon storage at ambient conditions for extended periods of time.

Thus, an aspect of the present invention relates to a fat encapsulated microbial culture comprising:
i) a preparation comprising a microbial culture, and
ii) an encapsulation matrix comprising one or more fat components, each of which fat components having a melting point of at least 25° C.

An embodiment of the present invention relates to the fat encapsulated microbial culture as described herein, wherein the one or more fat components have a melting point of at least 30° C., such as at least 31° C., such as at least 32° C., such as at least 33° C.

The fat components of the encapsulation matrix are characterised by their high melting point making them solid or semi-solid state at room temperature. This differentiates the fat components from most oils, such as olive oil, which are fluid at room temperature. The encapsulation matrix may comprise several fat components all of which have high melting points, with some variants of the encapsulation matrix having one fat component with especially high melting point. Such a fat component may be a hydrogenated or interesterified oil.

Another embodiment of the present invention relates to the fat encapsulated microbial culture as described herein, wherein at least one of the fat components have a melting point of at least 40° C., such as at least 45° C., such as at least 50° C., such as at least 55° C.

Alternatively, the encapsulation matrix may be defined by a common melting point representing an average melting point of all fat components of the encapsulation matrix. Such a definition may be preferred in situations where the choice of fat components is such that significant autonomous interesterification occurs between fat components resulting in change of the melting points of individual fat components. Therefore, an embodiment of the present invention relates to the fat encapsulated microbial culture as described herein, wherein the average melting point of the fat components is at least 25° C., such as at least 30° C., such as at least 31° C., such as at least 32° C., such as at least 33° C.

Two main parameters are affecting the melting point of fat components; the degree of saturation of the fatty acids and the carbon chain length. Double bonds introduce disorder in the packing of the fatty acids which leads to weaker inter-chain interactions and ultimately a lower melting temperature of unsaturated fat components. Similarly, shorter carbon chains allow less interactions than longer carbon chains, leading to fat components of increasing chain lengths having increasing melting points. Thus, favoured for the encapsulation matrix used to entrap the microbial culture are fatty acids with high degree of saturation and/or long carbon chain length.

Accordingly, an embodiment of the present invention relates to the fat encapsulated microbial culture as described herein, wherein the one or more fat components comprise at least 50 wt % of saturated fatty acids with respect to the total content of fatty acids of said one or more fat components.

Another embodiment of the present invention relates to the fat encapsulated microbial culture as described herein, wherein the one or more fat components comprise at least 60 wt % of saturated fatty acids with respect to the total content of fatty acids of said the one or more fat components.

Yet another embodiment of the present invention relates to the fat encapsulated microbial culture as described herein, wherein the one or more fat components comprise more than 50 wt % of fatty acids with a maximum carbon chain length of 16 carbon atoms with respect to the total content of fatty acids of the one or more fat components.

Fat components will in most instances comprise a mixture of fatty acids. These fatty acids are preferably selected among fatty acids with a high degree of saturation and/or long carbon chain length. Thus, an embodiment of the present invention relates to the fat encapsulated microbial culture as described herein, wherein the one or more fat components comprise at least 50 wt %, such as at least 60 wt %, such as at least 70 wt % of fatty acids selected from the group consisting of capric acid, lauric acid, myristic acid, palmitic acid, stearic acid, arachidic acid, behenic acid, lignoceric acid, cerotic acid, elaidic acid, vaccenic acid, erucic acid, and combinations thereof.

Another embodiment of the present invention relates to the fat encapsulated microbial culture as described herein, wherein the one or more fat components are selected from the group consisting of butterfat, cocoa butter, coconut fat, shea butter, mango kernel fat, palm oil, palm kernel oil, lard, hydrogenated oils, interesterified oils, and combinations thereof.

Of special interest is butterfat (also known as milk fat) because of is high degree of biocompatibility with a long range of products, such as dairy products. Therefore, an embodiment of the present invention relates to the fat encapsulated microbial culture as described herein, wherein the one or more fat components comprises butterfat. Another embodiment of the present invention relates to the fat encapsulated microbial culture as described herein, wherein the one or more fat components comprises butterfat and at least one additional fat component. Yet another embodiment of the present invention relates to the fat encapsulated microbial culture as described herein, wherein the one or more fat components comprise more than 60 wt % of fatty acids with a carbon chain length of 18 carbon atoms or less with respect to the total content of fatty acids of the one or more fat components.

Another group of fat components of interest are interesterified oils and hydrogenated oils due to their high melting points. The interesterified oils and hydrogenated oils may be provided as natural products or as synthetic products produced by hydrogenation of selected fatty acid and/or by interesterification by use of an enzyme, such as a lipase. Accordingly, an embodiment of the present invention relates to the fat encapsulated microbial culture as described herein, wherein the one or more fat components comprises interesterified oils and/or hydrogenated oils. Another embodiment of the present invention relates to the fat encapsulated microbial culture as described herein, wherein the one or more fat components is selected from interesterified oils and/or hydrogenated oils.

Hydrogenated oils suitable as fat components of the encapsulation matrix include, but are not limited to, hydrogenated rapeseed oil and hydrogenated coco-glycerides. Therefore, an embodiment of the present invention relates to the fat encapsulated microbial culture as described herein, wherein the one or more fat components comprises hydrogenated rapeseed oil and/or hydrogenated coco-glycerides.

For many practical purposes it may be beneficial to combine two or more fat components to formulate an encapsulation matrix with pre-determined characteristics directed at the specific type of product. Therefore, an embodiment of the present invention relates to the fat encapsulated microbial culture as described herein, wherein the encapsulation matrix comprises at least two fat components.

Another embodiment of the present invention relates to the fat encapsulated microbial culture as described herein, wherein the at least two fat components comprise hydrogenated coco-glycerides and hydrogenated rapeseed oil, respectively.

Emulsifiers may be included in the encapsulation matrix to reduce the viscosity and/or enhance blending of the fat components. Thus, an embodiment of the present invention relates to the fat encapsulated microbial culture as described herein, wherein the encapsulation matrix further comprises at least one emulsifier.

The emulsifier may be any traditional food-grade emulsifier that is compatible with the fat components of the encapsulation matrix. Accordingly, an embodiment of the present invention relates to the fat encapsulated microbial culture as described herein, wherein the at least one emulsifier is selected from the group consisting of monoglycerides, diglycerides, triglycerides, lecithin, milk proteins, gums and

Another embodiment of the present invention relates to the fat encapsulated microbial culture as described herein, wherein the emulsifier is polyglycerol polyricinoleate (PGPR).

The relative amounts of fat components to emulsifier can be varied to optimise flowability and blending of the encapsulation matrix. Thus, an embodiment of the present invention relates to the fat encapsulated microbial culture as described herein, wherein the ratio (wt %/wt %) of total fat components to emulsifier is in the range of 200:1 to 5:1, such as in the range of 100:1 to 5:1, such as in the range of 40:1 to 5:1, such as in the range of 30:1 to 10:1, preferably in the range of 25:1 to 15:1.

The ratio (wt %/wt %) of microbial culture to the encapsulation matrix may be customized to suit the specific application. It is contemplated that increasing the amount of encapsulation matrix may improve protection of the microbial culture from the environment. However, increasing the content of encapsulation matrix will dilute the content of microbial culture in the final product. Consequently, the optimal balance between microbial culture and encapsulation matrix is decided also by factors such as the time scale of storage and storage conditions, which may vary depending on the intended application. An embodiment of the present invention relates to the fat encapsulated microbial culture as described herein, wherein the ratio (wt %/wt %) of the microbial culture to the encapsulation matrix is in the range of 1:100 to 1:4, such as in the range of 1:50 to 1:4, such as in the range of 1:20 to 1:4, such as in the range of 1:15 to 1:6, preferably in the range of 1:12 to 1:8.

The microbial culture is typical provided in dried powder form, which allows storage of the microbial culture for extended periods of time before utilization in a final application. Keeping the water activity of the dried preparation low will contribute to the dry preparation not being spoiled over time. In general, a water activity of less than 0.6 should avoid any unwanted microorganisms to proliferate. The dried powder preparation comprising microbial culture may be obtained using any suitable method which does not significantly diminish viability of the microbial culture. Thus, an embodiment of the present invention relates to the fat encapsulated microbial culture as described herein, wherein the preparation is a dry preparation.

Another embodiment of the present invention relates to the fat encapsulated microbial culture as described herein, wherein the preparation has a water activity of less than 0.6 Aw, preferably less than 0.4 Aw, more preferably less than 0.3 Aw, most preferably less than 0.2 Aw. Yet another embodiment of the present invention relates to the fat encapsulated microbial culture as described herein, wherein the preparation has a water activity of less than 0.2 Aw, preferably less than 0.15 Aw.

A further embodiment of the present invention relates to the fat encapsulated microbial culture as described herein, wherein the preparation is selected from the group consisting of a freeze-dried, spray dried, vacuum dried and air-dried preparation. A still further embodiment of the present invention relates to the fat encapsulated microbial culture as described herein, wherein the preparation is a freeze-dried preparation.

The preparation comprising the microbial culture may be provided with one or more additives that benefit the preservation of the microbial culture before and/or after entrapment in the encapsulation matrix. Additives include, but are not limited to, agents that serves as cryoprotectants and antioxidants. Therefore, an embodiment of the present invention relates to the fat encapsulated microbial culture as described herein, wherein the preparation comprises one or more additives.

Another embodiment of the present invention relates to the fat encapsulated microbial culture as described herein, wherein the one or more additives are selected from the group consisting of antioxidants, carbohydrates, proteins, and combinations thereof.

Oxidation is the loss of electrons of an atom or ion. In the present context, oxidation refers to oxidation of molecular oxygen and means that oxygen is metabolised to unstable free radicals, which can pry away electrons from other molecules. Oxidation may therefore lead to damaging of cell membranes and other cellular components, such as proteins, lipids and DNA. To avoid damage to the fat encapsulated microbial culture, one or more antioxidants may be included in the preparation comprising the microbial culture to prevent oxidation. The antioxidants may be of either natural or synthetic origin.

An embodiment of the present invention relates to the fat encapsulated microbial culture as described herein, wherein the antioxidants are selected from the group consisting of citrate, ascorbate, tocopherol, ascorbyl palmitate, quercetin, gallic acid, tocotriene, tocotrienol, glutathione, and combinations thereof.

Another embodiment of the present invention relates to the fat encapsulated microbial culture as described herein, wherein the antioxidants are selected from vitamin C and/or vitamin E. It is to be understood that antioxidants as used herein include mineral salts of vitamin C, such as sodium ascorbate. Also, the vitamin E is to be understood as including all variants of tocopherols and tocotrienols (alpha, beta, gamma, delta).

Yet another embodiment of the present invention relates to the fat encapsulated microbial culture as described herein, wherein the antioxidants are selected from trisodium citrate and/or sodium ascorbate.

A further embodiment of the present invention relates to the fat encapsulated microbial culture as described herein, wherein the antioxidants are water-soluble or fat-soluble.

Cryoprotectants are used to improve the ability of microbial culture concentrates to survive against the harmful effect of freezing, frozen storage and freeze-drying. Preferably, these cryoprotectants should not be metabolized by the microbial strain to produce acids as it may cause a loss of viability due to damage to ATPase membrane-bound enzymes, (3-galactosidase, and cell membrane fluidity. In general, cryoprotectants that are not producing acids are more effective in improving the survival rate of freeze-dried microbial cultures. One preferred category of cryoprotectants is carbohydrates and the associated subgroups thereof.

Therefore, an embodiment of the present invention relates to the fat encapsulated microbial culture as described herein, wherein the carbohydrates are selected from the group consisting of monosaccharides, disaccharides, oligosaccharides, polysaccharides, and combinations thereof.

Another embodiment of the present invention relates to the fat encapsulated microbial culture as described herein, wherein the monosaccharides are selected from the group consisting of glucose, fructose, galactose, fucose, xylose, erythrose, and combinations thereof.

Yet another embodiment of the present invention relates to the fat encapsulated microbial culture as described herein, wherein the disaccharides are selected from the group consisting of lactose, sucrose, maltose, trehalose, cellobiose, and

A further embodiment of the present invention relates to the fat encapsulated microbial culture as described herein, wherein the oligosaccharides are selected from the group consisting of fructo-oligosaccharides (FOS), galactooligosaccharides (GOS), mannan oligosaccharides (MOS), and combinations thereof.

A still further embodiment of the present invention relates to the fat encapsulated microbial culture as described herein, wherein the polysaccharides are selected from the group consisting of maltodextrin, cellodextrin, gums, alginate, starch, glycogen, cellulose, chitin, pectin, inulin, dextran, carrageenan, chitosan, and combinations thereof.

An even further embodiment of the present invention relates to the fat encapsulated microbial culture as described herein, wherein the gums are selected from the group consisting of gum arabic, agar, alginate, cassia, dammar, pectin, beta-glucan, glucomannan, mastic, chicle, psyllium, spruce, gellan, guar, locust bean, xanthan, and combinations thereof. An embodiment of the present invention relates to the fat encapsulated microbial culture as described herein, wherein the gum is gum arabic.

Proteins may also be used as cryoprotectants Thus, an embodiment of the present invention relates to the fat encapsulated microbial culture as described herein, wherein the proteins are selected from the group consisting of caseinate, whey proteins, gelatine, plant proteins such as pea protein, potato protein, and rice protein, and combinations thereof.

The preparation to be encapsulated in the fat encapsulation matrix may principally comprise any type of microbial culture. Thus, the fat encapsulation technique as presented herein is not limited to a specific type of microbial culture but is a general fat encapsulation concept. Thus, it is contemplated that any type of microbial culture may advantageously be fat encapsulated as described herein.

Two types of microorganisms that are of great importance in many consumer goods are bacteria and yeast. These microorganisms are included e.g. in fermented food, feed mixes and nutritional supplements, wherein their health benefits are well-documented.

Therefore, an embodiment of the present invention relates to the fat encapsulated microbial culture as described herein, wherein the microbial culture is a bacterium or a yeast.

Another embodiment of the present invention relates to the fat encapsulated microbial culture as described herein, wherein the microbial culture is of a genus selected from the group consisting of Lactobacillus, Leuconostoc, Pediococcus, Lactococcus, Streptococcus, Enterococcus, Bifidobacterium, Propionibacterium, Bacillus and Saccharomyces.

Probiotic culture are cultures of live microorganisms, which upon ingestion by a subject provide health benefits to the subject. Products comprising probiotic cultures include, but are not limited to, dairy products, feed and beverages. Thus, it is to be understood that the fat encapsulated microbial cultures described herein may be administered not only to humans but also animals, and even plants.

Therefore, an embodiment of the present invention relates to the fat encapsulated microbial culture as described herein, wherein the microbial culture is a probiotic culture.

Another embodiment of the present invention relates to the fat encapsulated microbial culture as described herein, wherein the probiotic culture is of a genus selected from the group consisting of Lactobacillus or Bifidobacterium.

Yet another embodiment of the present invention relates to the fat encapsulated microbial culture as described herein, wherein the probiotic culture is selected from the group consisting of Lacticaseibacillus rhamnosus, Ligilactobacillus animalis and Bifidobacterium animalis subsp. Lactis.

Of particular interest are lactic acid bacteria (LAB) that are an order of Gram-positive bacteria sharing common metabolic and physiological characteristics. LAB produce lactic acid as the major metabolic outcome of carbohydrate fermentation. Ever since it was discovered that acidification by food fermentation could preserve food by inhibiting growth of spoilage agents, LAB has been utilized purposefully in food fermentation. While post-acidification presents a challenge with regards to microbial culture viability, the fat encapsulation provided herein overcomes this issue. Without being bound by theory, it is contemplated that the encapsulation matrix act as a barrier to the diffusion of lactose from the lactose-containing product, such as yogurt, to inside of the fat microcapsules. Therefore, the metabolic activity of encapsulated microbial is low due to the limitation of substrate availability. Additionally, fat encapsulation also reduces the inactivation rate of the microbial culture, leading to maintenance of viability even at ambient storage conditions. Consequently, the fat encapsulated microbial cultures described herein tolerate both post pasteurisation as well as subsequent storage at ambient temperatures and may thus open up development of post pasteurised products containing microbial cultures, such as LAB, to a broader ensemble of product developers without the need for additional investment in the existing production line, such as installation of sterile in-line mixing.

Thus, an embodiment of the present invention relates to the fat encapsulated microbial culture as described herein, wherein the microbial culture is a lactic acid bacteria (LAB).

Another embodiment of the present invention relates to the microencapsulated microbial culture as described herein, wherein the microbial culture is or comprises a lactic acid bacteria (LAB) of a genus selected from the group consisting of Lactobacillus, Holzapfelia, Amylolactobacillus, Bombilactobacillus, Companilactobacillus, Lapidilactobacillus, Agrilactobacillus, Schleiferilactobacillus, Loigolactobacilus, Lacticaseibacillus, Latilactobacillus, Dellaglioa, Liguorilactobacillus, Ligilactobacillus, Lactiplantibacillus, Fulfurilactobacillus, Paucilactobacillus, Limosilactobacillus, Fructilactobacillus, Acetilactobacillus, Apilactobacillus, Levilactobacillus, Secundilactobacillus and Lentilactobacillus, Leuconostoc, Pediococcus, Lactococcus, Streptococcus, Enterococcus, Bifidobacterium, Brevibacterium, and Staphylococcus.

It will be appreciated that the Lactobacillus genus taxonomy was updated in 2020. The new taxonomy is disclosed in Zheng et al. 2020 and will be cohered to herein if nothing else is noticed. For the purpose of the present invention, table 1 presents a list of new and old names of some Lactobacillus species relevant to the present invention.

TABLE 1
New and old names of some Lactobacillus
species relevant to the present invention.

| Old Name | New Name |
|---|---|
| Lactobacillus reuteri | Limosilactobacillus reuteri |
| Lactobacillus rhamnosus | Lacticaseibacillus rhamnosus |
| Lactobacillus salivarius | Ligilactobacillus salivarius |
| Lactobacillus casei | Lacticaseibacillus casei |
| Lactobacillus paracasei | Lacticaseibacillus paracasei |
| subsp. paracasei | subsp. Paracasei |
| Lactobacillus plantarum | Lactiplantibacillus plantarum |
| subsp. plantarum | subsp. plantarum |
| Lactobacillus fermentum | Limosilactobacillus fermentum |
| Lactobacillus animalis | Ligilactobacillus animalis |
| Lactobacillus buchneri | Lentilactobacillus buchneri |
| Lactobacillus curvatus | Latilactobacillus curvatus |
| Lactobacillus futsaii | Companilactobacillus futsaii |
| Lactobacillus sakei subsp. sakei | Latilactobacillus sakei subsp. |
| Lactobacillus pentosus | Lactiplantibacillus pentosus |

Bacteria of the Lactobacillus genus, as well as the related newly updated genera, have for a long time been known to constitute a significant component of the microbiota in the human body, such as in the digestive system, urinary system and genital system. For this reason, these bacteria have been heavily utilized in in health and/or nutritional products aimed at aiding, maintaining or restoring the natural balance of microbiota in the human body. Examples of application of Lactobacillus include treatment or amelioration of diarrhea, vaginal infections, and skin disorders such as eczema.

Thus, an embodiment of the present invention relates to the microencapsulated microbial culture as described herein, wherein the microbial culture is or comprises a lactic acid bacteria (LAB) of a genus selected from the group consisting of Lactobacillus, Limosilactobacillus, Lacticaseibacillus, Ligilactobacillus, Lacticaseibacillus, Lacticaseibacillus, Lactiplantibacillus, Limosilactobacillus, Ligilactobacillus, Lentilactobacillus, Latilactobacillus, Companilactobacillus, Latilactobacillus and Lactiplantibacillus. Another embodiment of the present invention relates to the microencapsulated microbial culture as described herein, wherein the microbial culture is of a species of Limosilactobacillus reuteri, Lacticaseibacillus rhamnosus, Ligilactobacillus salivarius, Lacticaseibacillus casei, Lacticaseibacillus paracasei subsp. paracasei, Lactiplantibacillus plantarum subsp. plantarum, Limosilactobacillus fermenturn, Ligilactobacillus animalis, Lentilactobacillus buchneri, Latilactobacillus curvatus, Cornpanilactobacillus futsaii, Latilactobacillus sakei subsp., Lactiplantibacillus pentosus, Lactobacillus acidophillus, Lactobacillus helveticus, Lactobacillus gasseri and Lactobacillus delbrueckii.

A further embodiment of the present invention relates to the fat encapsulated microbial culture as described herein, wherein the preparation comprises two or more microbial cultures, preferably two microbial cultures.

Upon entrapment of the preparation comprising a microbial culture in the encapsulation matrix small microcapsules are formed. Without being bound by theory, the microcapsules can be characterised as spherical with an interior and exterior part. It may for some applications be desired to apply additional coatings on top of the exterior part of the microcapsules. Thus, an embodiment of the present invention relates to the fat encapsulated microbial culture as described herein, wherein the microbial culture is comprised in an interior part and the encapsulation matrix is comprised in an exterior part.

Another embodiment of the present invention relates to the fat encapsulated microbial culture as described herein, wherein the fat encapsulated microbial culture further comprises one or more coatings.

Yet another embodiment of the present invention relates to the fat encapsulated microbial culture as described herein further comprising a hydrophobic coating.

To significantly impact the nutritional value and aid the health of the consumer, it is preferred that the microbial culture is initially provided in sufficient amounts. Therefore, an embodiment of the present invention relates to the fat encapsulated microbial culture as described herein, wherein the preparation comprises in the range of 1.0E+07 to 5.0E+11 CFU/g microbial culture, preferably in the range of 1.0E+09 to 1.0E+11 CFU/g microbial culture, more preferably in the range of 1.0E+10 to CFU/g microbial culture.

Another embodiment of the present invention relates to the fat encapsulated microbial culture as described herein, wherein the preparation comprises one or more yield enhancing agents selected from the group consisting of a purine base, a pyrimidine base, a nucleoside, a nucleotide and derivatives thereof.

For most practical purposes, the fat encapsulated microbial cultures described herein will be included in a composition comprising further ingredients as part of final processing of the end product. That end product may be any product which would provide the recipient with a beneficial effect from the inclusion of a microbial culture, such as a probiotic culture.

Thus, an embodiment of the present invention relates to a composition comprising the fat encapsulated microbial culture as described herein.

Another embodiment of the present invention relates to the composition as described herein, wherein the composition is selected from the group consisting of a feed, a plant health product, a food, a beverage and a pharmaceutical product.

Another aspect of the present invention relates to a product comprising a fat encapsulated microbial culture or a composition as described herein, wherein said product is selected from the group consisting of a feed, a plant health product, a food, a beverage and a pharmaceutical product.

It is to be understood that the term “food” includes also “extruded food products” and “bars”, and that “food” or “feed” may be in the form of a pre-mix that is intended to be further mixed with additional ingredients to obtain a final product. Thus, an embodiment of the present invention relates to the product or the composition as described herein, wherein the product or composition is a pre-mix. Another embodiment of the present invention relates to the product or the composition as described herein, wherein the product or composition is an extruded food product or a bar. A bar is a texturized product made by extrusion where different food components are held together with edible adhesives, such as, but not limited to, sugars.

A preferred type of food are dairy products, which are widely consumed worldwide and considered vehicle or source for delivery of beneficial microbial cultures, such as probiotics. Thus, an aspect of the present invention relates to a dairy product comprising a fat encapsulated microbial culture or a composition as described herein.

Another embodiment of the present invention relates to the dairy product as described herein, wherein said dairy product is selected from the group consisting of yoghurt, cheese, butter, an inoculated sweet milk and a liquid fermented milk product.

A milk-based product such as yogurt is a well characterized carrier suitable to protect and deliver microbial cultures, such as probiotics, in the gut. One of the reasons is that yogurt is rich in nutrients, proteins, fatty acids, carbohydrates, vitamins, minerals, and calcium, which improves the capability of the probiotic strains to bind the epithelial cell. Another reason is that the consumer considers yogurt a nutritional, healthy, and natural carrier of living bacteria, so preferably consumes it daily.

Yogurt is a semi-solid fermented product made from heat-treated standardized milk. Typically, the main starter cultures or yogurt cultures are used for making yogurt, and probiotic bacteria are added as adjunct cultures. For post-pasteurised yoghurts (PPY) these microbial cultures are challenged to withstand environmental stress such as high temperature and post-acidification.

Yoghurts tend to have pH values in the range of approximately 4.0 to 4.6 as fermentation comes to an end, but any further post-acidification may cause a decline in viable cell counts and impair the consumer experience.

Moreover, microbial cultures, such as probiotics, incorporated in post-pasteurized yogurt goes through a very high heating step, which challenges their survivability. Typical heat treatment in the post pasteurized unit is 72° C. for 20 seconds (approx. 63° C. for 30 minutes at low pasteurization).

The fat encapsulated microbial culture provided herein can withstand the challenges relating to heat stress and post-acidification even at ambient temperature. This can potentially lead to new products for the PPY market where viable microbial cells, such as probiotic cells, are needed after post-pasteurization. Importantly, the microbial cells can be added to the milk just before post-pasteurization. This means that the manufacturers of PPY do not have to invest in any new technology for aseptically adding the microbial culture after post-pasteurization.

Therefore, an embodiment of the present invention relates to the dairy product as described herein, wherein said dairy product is a post-pasteurized yoghurt (PPY).

It is to be understood that the term “yoghurt” as used herein include in addition to conventional yoghurt also special variants of yoghurt, such as local/regional yoghurts prepared through special processing, sweetened and/or flavoured yoghurts, strained yoghurts, yoghurt-based beverages, and plant-based yoghurt (also termed plant-milk yoghurts). Plant-based yoghurts include, but are not limited to, yoghurts based on soy milk, rice milk, and nut milks such as almond milk and coconut milk fermented with yogurt cultures. Plant-based yoghurts may be suitable for vegans, people with intolerance to dairy milk, and/or those who prefer plant-based products for e.g. political or environmental reasons. Therefore, an embodiment of the present invention relates to the dairy product as described herein, wherein said dairy product is a yoghurt, such as a post-pasteurized yoghurt, selected from the group consisting of yoghurts prepared through special processing, sweetened yoghurts, flavoured yoghurts, strained yoghurts, yoghurt-based beverages and plant-based yoghurt. Another embodiment of the present invention relates to the dairy product as described herein, wherein said dairy product is a plant-based yoghurt.

The composition or dairy product described herein may comprise one or more food-grade ingredients, which are compound that is non-toxic and safe for consumption and comply with the Food Chemicals Codex (FCC). Food-grade ingredients include, but are not limited to, compounds that can alter attributes such as aroma, flavour, acidity, colour, viscosity and texture, as well as preservatives, nutrients, thickeners, sweeteners and emulsifiers. Therefore, an embodiment of the present invention relates to the composition or dairy product as described herein further comprising one or more food-grade ingredients.

Another embodiment of the present invention relates to the composition or dairy product as described herein, wherein the one or more food-grade ingredients are selected from the group consisting of compounds that can alter attributes such as aroma, flavour, acidity, colour, viscosity and texture, as well as preservatives, nutrients, thickeners, sweeteners, emulsifiers, and combinations thereof.

Preferred food-grade ingredients include, but are not limited to, lactose, maltodextrin, whey protein, casein, corn starch, dietary fibres, gums and gelatine. Thus, an embodiment of the present invention relates to the composition or dairy product as described herein, wherein the one or more food-grade ingredients are selected from the group consisting of lactose, maltodextrin, whey protein, casein, corn starch, dietary fibres, gums, gelatine, and combinations thereof.

To benefit the health of the consumer it is important that the end-product comprise a significant of viable microbial cells. These will contribute to the functions regulated by the natural population of live microbes within the consumer, such as in the intestines. Since dairy products that are post-pasteurized are especially suitable for distribution to areas where cooling is not possible, it is also a requirement that the microbial cultures contained within such product are capable of surviving storage under environmental stress, such as elevated (non-refrigerated) temperatures. Without being bound by theory, the fat coatings seem to reduce the inactivation rate of the entrapped microbial culture, ultimately leading to maintenance of their viability even at ambient storage conditions. Therefore, an embodiment of the present invention relates to the dairy product as described herein, wherein said PPY comprises 1.0E+05 to 5.0E+09 CFU/g microbial culture, preferably in the range of 1.0E+07 to 1.0E+09 CFU/g microbial culture, more preferably in the range of 1.0E+08 to 5.0E+08 CFU/g microbial culture.

Another embodiment of the present invention relates to the dairy product as described herein, wherein the loss in viability of the microbial culture as measured by CFU/g is less than 2 log units after storage for 2 months at 25° C., preferably less than 1 log unit after storage for 2 months at 25° C., more preferably less than 0.5 log unit after storage for 2 months at 25° C.

The fat encapsulated microbial cultures described herein are efficient in avoiding any post-acidification subsequent to deliberate fermentation of the product. This is demonstrated both by low viability loss over extended storage and by direct measurement of the pH within model yoghurt showing stable pH values over time. Without being bound by theory, the low post-acidification of the fat encapsulated microbial cultures indicates that fat encapsulation slows down the strain's capacity to produce lactic acid. It is contemplated that this may be caused by the fat coating acting as a barrier to the diffusion of lactose from the yoghurt to inside of the microcapsules. As a consequence, the metabolic activity of the encapsulated microbial culture is low due to the limitation of substrate availability.

Thus, an embodiment of the present invention relates to the dairy product as described herein, wherein the pH of the dairy product is maintained in the range of pH 3.8 to pH 5.0 after storage for 2 months at 25° C., such as in the range of pH 4.1. to pH 4.7 after storage for 2 months at 25° C., such as in the range of pH 4.2. to pH 4.6, preferably in the range of pH 4.3. to pH 4.5.

The microbial cultures are encapsulated by applying an encapsulation matrix comprising one or more fat components to a preparation comprising the microbial culture. Thus, an aspect of the present invention relates to a method for preparing a fat encapsulated microbial culture or a composition as described herein, said method comprising the steps of:
i) provision of a preparation comprising a microbial culture,
ii) provision of an encapsulation matrix, and
iii) mixing the encapsulation matrix with the preparation to form a microencapsulated microbial culture,

wherein the encapsulation matrix comprises one or more fat components, each of which fat components having a melting point of at least 25° C.

Typically, the encapsulation matrix is provided in fluid state to facilitate homogeneous coating of the microbial culture. Accordingly, the temperature of the blending process may be adjusted according to the melting point of the fat components in the encapsulation matrix. Thus, an embodiment of the present invention relates to the method as described herein, wherein the encapsulation matrix of step ii) is provided at a temperature above the melting temperature of each of the fat components to provide a molten encapsulation matrix.

Another embodiment of the present invention relates to the method as described herein, wherein the temperature of step ii) is at least 40° C., such as at least 50° C., such as at least 60° C., preferably at least 70° C.

The present method for fat encapsulation of a microbial culture is not limited to any specific type of microbial culture, nor any specific physical state of the microbial culture. However, many microbial cultures are out of convenience dried during processing, e.g. as a last step after culturing of the microorganisms. Drying of the microbial culture may ease downstream handling and extend the shelf life of the microbial culture.

Therefore, an embodiment of the present invention relates to the method as described herein, wherein the preparation of step i) is provided as a dry preparation.

Another embodiment of the present invention relates to the method as describer herein, wherein the dry preparation is selected from the group consisting of a freeze-dried, spray dried, vacuum dried and air-dried preparation.

Yet another embodiment of the present invention relates to the method as described herein, wherein the dry preparation is a freeze-dried preparation.

Process parameters may be adjusted to optimise entrapment of the microbial culture in the fat coating without compromising the viability of the cells.

Therefore, an embodiment of the present invention relates to the method as described herein, wherein the dry preparation is kept at a temperature of at least 20° C., such as in the range of 20° C. to 30° C., for at least 20 min, such as 30 min, prior to step iii).

Another embodiment of the present invention relates to the method as described herein, wherein mixing in step iii) is performed for at least 10 seconds.

The preparation comprising a microbial culture can also be coated with a fat blend by using a fluid-bed coater, such as Mini Glatt. In this case the molten fat blend is prepared in the same way as described in Example 2 herein. Next, the preparation comprising a microbial culture (e.g. as a freeze-dried powder) is filled on top of the sieve in the Mini Glatt which is fitted with a two-fluid nozzle, the Wurster set-up and the micro insert for handling small powder samples. The preparation comprising a microbial culture is then fluidized. The fluidized-bed coating process is carried out until a desired level of fat coating e.g. 35% w/w has been achieved. The microparticles can be collected and further used as described in Example 2 herein.

Fluidization in the fluid-bed coater may be performed with a gas flow rate of 20 m3/h with inlet temperature of 50° C. and outlet temperature of 35° C. Other suitable parameters include, but are not limited to, a pressure drop across the plate is around 10-15 mBar, with the nozzle pressure being set to around 2 Bar and the material being sprayed at a rate of 5-10 g/min. The filter cleaning pressure may be set to 3 Bar.

As an alternative to pelletization in liquid nitrogen, final microparticles may be obtained by the method of spray chilling. In this case the preliminary steps are same as described in Example 2 herein. The main difference is that instead of pelletizing in liquid nitrogen, the suspension of microbial culture in the molten fat blend is atomized into a cold chamber (temperature of approximately 10-15° C.) using a two-fluid nozzle (e.g. with a 0.7 mm diameter) and atomized. The atomization air pressure may be approximately 1 kPa. The microparticles can be collected and further used as described in example 2.

Upon preparation of a yoghurt, a starter culture is added to milk to initiate conversion of lactose into lactic acid. The acidic environment causes the casein protein of the milk to coagulate and thicken the milk to the well-known yoghurt texture. Besides lactic acid, a yoghurt comprises also acetaldehyde, acetic acid and diacetyl acid, all of which together gives the yoghurt the characteristics tartness, taste and flavour as well as aid in the development of a strong immune system. Accordingly, the acidic environment plays an important role for preparation of yoghurts, but also presents a challenge for provision of viable microbial cultures, such as probiotics, as microbial growth is typically hindered if the pH drops too low.

Of particular interest herein are therefore methods for producing post-pasteurised yoghurts (PPY) comprising high amounts of viable beneficial microorganisms, such as probiotic cultures. Thus, an aspect of the present invention relates to a method for preparing a post-pasteurized yoghurt (PPY), said method comprising the steps of:
i) provision of a yoghurt with a pH in the range of 4.0-4.6,
ii) addition of a fat encapsulated microbial culture or a composition as described herein to the yoghurt, and
iii) post-pasteurization of the yoghurt of step ii).

The pH of the yoghurt may be selected to optimise quality, consistency and taste as well as to improve shelf life. Thus, an embodiment of the present invention relates to the method as described herein, wherein the yoghurt of step i) has a pH in the range of 4.3-4.5.

Another embodiment of the present invention relates to the method as described herein, wherein the yoghurt of step i) is a pasteurized yoghurt.

The first (initial) pasteurisation and the second (post) pasteurisation steps may be performed as either batch pasteurisation or continuous pasteurisation. Batch pasteurization is the processing of limited volumes of fluid, such as milk, in a container at a specific temperature for a long period of time. The milk is heated a held under agitation throughout a holding period. Upon completion of the holding time, the milk may be either cooled in the container or removed hot. Continuous pasteurisation on the other hand process a stream of fluid, such as milk, using e.g. a high temperature short time (HTST) pasteurizer. Heating of the milk is performed in channels defined by gaskets and mediated by a heating medium such as vacuum steam or hot water.

Thus, an embodiment of the present invention relates to the method as described herein, wherein the post-pasteurization of step iii) is performed by batch pasteurisation or continuous pasteurisation.

Another embodiment of the present invention relates to the method as described herein, wherein the post-pasteurization of step iii) is performed at a temperature of at least 60° C.

Yet another embodiment of the present invention relates to the method as described herein, wherein the post-pasteurization of step iii) is performed for at least 20 min, such as for 30 min.

A further embodiment of the present invention relates to the method as described herein, wherein the post-pasteurization of step iii) is performed at a temperature in the range of 65-75° C. for time period in the range of 2-10 minutes.

A still further embodiment of the present invention relates to the method as described herein, wherein the post-pasteurization of step iii) is performed at a temperature of 70° C. for 5 minutes.

It is to be understood that higher pasteurization temperatures will allow shorter time and vice versa. Thus, a person skilled in the art would know how to adjust the temperature and time of pasteurisation to be suitable for the method described herein.

Importantly, the content of microbial culture in the final PPY should be high enough to provide a beneficial health and nutritional effect. The main challenges to achieve this is to provide a microbial culture that is capable of surviving the post-pasteurisation step and avoid subsequent post-acidification upon storage. These challenges are solved by the method described herein by fat encapsulation of the microbial culture.

Therefore, an embodiment of the present invention relates to the method as described herein, wherein the content of microbial culture in the PPY is in the range of 1.0E+05 to 5.0E+09 CFU/g, preferably in the range of 1.0E+06 to 5.0E+08 CFU/g.

Another embodiment of the present invention relates to the method as described herein, wherein the loss in viability of the microbial culture in the PPY as measured by CFU/g is less than 2 log units after storage for 2 months at 25° C., preferably less than 1 log unit after storage for 2 months at 25° C., more preferably less than 0.5 log unit after storage for 2 months at 25° C.

A further embodiment of the present invention relates to the method as described herein, wherein the pH of the PPY is maintained in the range of pH 3.8 to pH 5.0 after storage for 2 months at 25° C., such as in the range of pH 4.1. to pH 4.7 after storage for 2 months at 25° C., such as in the range of pH 4.2. to pH 4.6, preferably in the range of pH 4.3. to pH 4.5.

The methods described herein are suitable for obtaining microbial cultures entrapped within a fat coating. These improved microbial cultures may be used as additives or as part of more complex compositions in a wide range of products wherein they provide health and nutritional benefits to the consumer, such as improved immune system and enhanced digestion. The consumer is not limited to humans, but could also be animals, such as domesticated animals.

Accordingly, an aspect of the present invention relates to a fat encapsulated microbial culture or a composition as described herein obtainable by a method as described herein.

Another aspect of the present invention relates to a product comprising a fat encapsulated microbial culture or a composition as described herein, wherein said product is selected from the group consisting of a feed, a plant health product, a food, a beverage and a pharmaceutical product.

Preferred products wherein it would be suitable to include the fat encapsulated microbial cultures as an additive include a range of different dairy products. Thus, an aspect of the present invention relates to a dairy product comprising a fat encapsulated microbial culture or a composition as described herein, wherein said dairy product is selected from the group consisting of yoghurt, cheese, butter, an inoculated sweet milk and a liquid fermented milk product.

An embodiment of the present invention relates to the dairy product as described herein, wherein said dairy product is a post-pasteurized yoghurt (PPY).

The fat encapsulated microbial culture may be used as a stand-alone additive or as a component in a composition comprising further ingredients. Accordingly, an aspect of the present invention relates to use of a fat encapsulated microbial culture or a composition as described herein in a product selected from the group consisting of a feed, a plant health product, a food, a beverage and/or a pharmaceutical product.

An embodiment of the present invention relates to use of a fat encapsulated microbial culture or a composition as described herein in a dairy product selected from the group consisting of yoghurt, cheese, butter, an inoculated sweet milk and a liquid fermented milk product.

Another embodiment of the present invention relates to the use as described herein, wherein said dairy product is a post-pasteurized yoghurt (PPY).

The listing or discussion of an apparently prior published document in this specification should not necessarily be taken as an acknowledgement that the document is part of the state of the art or is common general knowledge.

Preferences, options and embodiments for a given aspect, feature or parameter of the invention should, unless the context indicates otherwise, be regarded as having been disclosed in combination with any and all preferences, options and embodiments for all other aspects, features and parameters of the invention. This is especially true for the description of the fat encapsulated microbial culture and all its features, which may readily be part of the final composition or product obtained by the method as described herein. Embodiments and features of the present invention are also outlined in the following items.

Items
X1. A fat encapsulated microbial culture comprising:
   i) a preparation comprising a microbial culture, and
   ii) an encapsulation matrix comprising one or more fat components, each of which fat components having a melting point of at least 25° C.
X2. The fat encapsulated microbial culture according to item X1, wherein the one or more fat components have a melting point of at least 30° C., such as at least 31° C., such as at least 32° C., such as at least 33° C.
X3. The fat encapsulated microbial culture according to any one of items X1 or X2, wherein at least one of the fat components have a melting point of at least 40° C., such as at least 45° C., such as at least 50° C., such as at least 55° C.
X4. The fat encapsulated microbial culture according to any one of the preceding items, wherein the one or more fat components comprise at least 50 wt % of saturated fatty acids with respect to the total content of fatty acids of said one or more fat components.
X5. The fat encapsulated microbial culture according to any one of the preceding items, wherein the one or more fat components comprise at least 60 wt % of saturated fatty acids with respect to the total content of fatty acids of said the one or more fat components.
X6. The fat encapsulated microbial culture according to any one of the preceding items, wherein the one or more fat components comprise more than 50 wt % of fatty acids with a maximum carbon chain length of 16 carbon atoms with respect to the total content of fatty acids of the one or more fat components.
X7. The fat encapsulated microbial culture according to any one of the preceding items, wherein the one or more fat components are selected from the group consisting of butterfat, cocoa butter, coconut fat, shea butter, mango kernel fat, palm oil, palm kernel oil, lard, hydrogenated oils, interesterified oils, and combinations thereof.
X8. The fat encapsulated microbial culture according to any one of the preceding items, wherein the encapsulation matrix comprises at least two fat components.
X9. The fat encapsulated microbial culture according to item X8, wherein the at least two fat components comprise hydrogenated coco-glycerides and hydrogenated rapeseed oil, respectively.
X10. The fat encapsulated microbial culture according to any one of the preceding items, wherein the encapsulation matrix further comprises at least one emulsifier.
X11. The fat encapsulated microbial culture according to item X10, wherein the at least one emulsifier is selected from the group consisting of monoglycerides, diglycerides, triglycerides, lecithin, milk proteins, gums and combinations thereof.
X12. The fat encapsulated microbial culture according to any one of items X10 or X11, wherein the emulsifier is polyglycerol polyricinoleate (PGPR).
X13. The fat encapsulated microbial culture according to any one of items X10-X12, wherein the ratio (wt %/wt %) of total fat components to emulsifier is in the range of 200:1 to 5:1, such as in the range of 100:1 to 5:1, such as in the range of 40:1 to 5:1, such as in the range of 30:1 to 10:1, preferably in the range of 25:1 to 15:1.
X14. The fat encapsulated microbial culture according to any one of the preceding items, wherein the ratio (wt %/wt %) of the microbial culture to the encapsulation matrix is in the range of 1:100 to 1:4, such as in the range of 1:50 to 1:4, such as in the range of 1:20 to 1:4, such as in the range of 1:15 to 1:6, preferably in the range of 1:12 to 1:8.
X15. The fat encapsulated microbial culture according to any one of the preceding items, wherein the preparation is a dry preparation.
X16. The fat encapsulated microbial culture according to any one of the preceding items, wherein the preparation has a water activity of less than 0.2 Aw, preferably less than 0.15 Aw.
X17. The fat encapsulated microbial culture according to any one of the preceding items, wherein the preparation is selected from the group consisting of a freeze-dried, spray dried, vacuum dried and air-dried preparation.
X18. The fat encapsulated microbial culture according to any one of the preceding items, wherein the preparation is a freeze-dried preparation.
X19. The fat encapsulated microbial culture according to any one of the preceding items, wherein the preparation comprises one or more additives.
X20. The fat encapsulated microbial culture according to item X19, wherein the one or more additives are selected from the group consisting of antioxidants, carbohydrates, proteins, and combinations thereof.
X21. The fat encapsulated microbial culture according to item X20, wherein the antioxidants are selected from the group consisting of citrate, ascorbate, tocopherol, ascorbyl palmitate, quercetin, gallic acid, tocotriene, tocotrienol, glutathione, and combinations thereof.
X22. The fat encapsulated microbial culture according to items X20 or X21, wherein the carbohydrates are selected from the group consisting of monosaccharides, disaccharides, oligosaccharides, polysaccharides, and combinations thereof.
X23. The fat encapsulated microbial culture according to item X22, wherein the monosaccharides are selected from the group consisting of glucose, fructose, galactose, fucose, xylose, erythrose, and combinations thereof.
X24. The fat encapsulated microbial culture according to any one of items X22 or X23, wherein the disaccharides are selected from the group consisting of lactose, sucrose, maltose, trehalose, cellobiose, and combinations thereof.
X25. The fat encapsulated microbial culture according to any one of items X22-X24, wherein the oligosaccharides are selected from the group consisting of fructo-oligosaccharides (FOS), galactooligosaccharides (GOS), mannan oligosaccharides (MOS), and combinations thereof.
X26. The fat encapsulated microbial culture according to any one of items X22-X25, wherein the polysaccharides are selected from the group consisting of maltodextrin, cellodextrin, gums, alginate, starch, glycogen, cellulose, chitin, pectin, inulin, dextran, carrageenan, chitosan, and combinations thereof.
X27. The fat encapsulated microbial culture according to item X26, wherein the gums are selected from the group consisting of gum arabic, agar, alginate, cassia, dammar, pectin, beta-glucan, glucomannan, mastic, chicle, psyllium, spruce, gellan, guar, locust bean, xanthan, and combinations thereof.
X28. The fat encapsulated microbial culture according to item X27, wherein the gum is gum arabic.
X29. The fat encapsulated microbial culture according to any one of items X20-X28, wherein the proteins are selected from the group consisting of caseinate, whey proteins, gelatine, plant proteins such as pea protein, potato protein, and rice protein, and combinations thereof.
X30. The fat encapsulated microbial culture according to any one of the preceding items, wherein the microbial culture is of a genus selected from the group consisting of Lactobacillus, Leuconostoc, Pediococcus, Lactococcus, Streptococcus, Enterococcus, Bifidobacterium, Propionibacterium, Bacillus and Saccharomyces.
X31. The fat encapsulated microbial culture according to any one of the preceding items, wherein the microbial culture is a probiotic culture.
X32. The fat encapsulated microbial culture according to item X31, wherein the probiotic culture is of a genus selected from the group consisting of Lactobacillus or Bifidobacterium.
X33. The fat encapsulated microbial culture according to any one of items X31 or X32, wherein the probiotic culture is selected from the group consisting of Lacticaseibacillus rhamnosus, Ligilactobacillus animalis and Bifidobacterium animalis subsp. Lactis.
X34. The fat encapsulated microbial culture according to any one of the preceding items, wherein the microbial culture is a lactic acid bacteria (LAB).
X35. The fat encapsulated microbial culture according to any one of the preceding items, wherein the microbial culture is comprised in an interior part and the encapsulation matrix is comprised in an exterior part.
X36. The fat encapsulated microbial culture according to any one of the preceding items, wherein the fat encapsulated microbial culture further comprises one or more coatings.
X37. The fat encapsulated microbial culture according to any one of the preceding items further comprising a hydrophobic coating.
X38. The fat encapsulated microbial culture according to any one of the preceding items, wherein the preparation comprises in the range of 1.0E+07 to 5.0E+11 CFU/g microbial culture, preferably in the range of 1.0E+09 to 1.0E+11 CFU/g microbial culture, more preferably in the range of 1.0E+10 to 5.0E+10 CFU/g microbial culture.
X39. The fat encapsulated microbial culture according to any one of the preceding items, wherein the preparation comprises one or more yield enhancing agents selected from the group consisting of a purine base, a pyrimidine base, a nucleoside, a nucleotide and derivatives thereof.
X40. A composition comprising the fat encapsulated microbial culture according to any one of the preceding items.
X41. The composition according to item X40, wherein the composition is selected from the group consisting of a feed, a plant health product, a food, a beverage and a pharmaceutical product.
X42. A dairy product comprising a fat encapsulated microbial culture according to any one of items X1-X39 or a composition according to any one of items X40 or X41.
X43. The dairy product according to item X42, wherein said dairy product is selected from the group consisting of yoghurt, cheese, butter, an inoculated sweet milk and a liquid fermented milk product.
X44. The dairy product according to any one of items X42 or X43, wherein said dairy product is a post-pasteurized yoghurt (PPY).
X45. The dairy product according to item X44, wherein said PPY comprises 1.0E+05 to 5.0E+09 CFU/g microbial culture, preferably in the range of 1.0E+07 to 1.0E+09 CFU/g microbial culture, more preferably in the range of 1.0E+08 to 5.0E+08 CFU/g microbial culture.
X46. The dairy product according to any one of items X42-X45, wherein the loss in viability of the microbial culture as measured by CFU/g is less than 2 log units after storage for 2 months at 25° C., preferably less than 1 log unit after storage for 2 months at 25° C., more preferably less than 0.5 log unit after storage for 2 months at
X47. The dairy product according to any one of items X42-X46, wherein the pH of the dairy product is maintained in the range of pH 3.8 to pH 5.0 after storage for 2 months at 25° C., such as in the range of pH 4.1. to pH 4.7 after storage for 2 months at 25° C., such as in the range of pH 4.2. to pH 4.6, preferably in the range of pH 4.3. to pH 4.5.
Y1. A method for preparing a fat encapsulated microbial culture according to any one of items X1-X39 or a composition according to any one of items X40 or X41, said method comprising the steps of:
   i) provision of a preparation comprising a microbial culture,
   ii) provision of an encapsulation matrix, and
   iii) mixing the encapsulation matrix with the preparation to form a microencapsulated microbial culture, wherein the encapsulation matrix comprises one or more fat components, each of which fat components having a melting point of at least 25° C.
Y2. The method according to item Y1, wherein the encapsulation matrix of step ii) is provided at a temperature above the melting temperature of each of the fat components to provide a molten encapsulation matrix.
Y3. The method according to item Y2, wherein the temperature of step ii) is at least 40° C., such as at least 50° C., such as at least 60° C., preferably at least 70° C.
Y4. The method according to any one of items Y1-Y3, wherein the preparation of step i) is provided as a dry preparation.
Y5. The method according to item Y4, wherein the dry preparation is selected from the group consisting of a freeze-dried, spray dried, vacuum dried and air-dried preparation.
Y6. The method according to any one of items Y4 or Y5, wherein the dry preparation is a freeze-dried preparation.
Y7. The method according to any one of items Y4-Y6, wherein the dry preparation is kept at a temperature of at least 20° C., such as in the range of 20° C. to 30° C., for at least 20 min, such as 30 min, prior to step iii).
Y8. The method according to any one of items Y1-Y7, wherein mixing in step iii) is performed for at least 10 seconds.
W1. A method for preparing a post-pasteurized yoghurt (PPY), said method comprising the steps of:
   i) provision of a yoghurt with a pH in the range of 4.0-4.6,
   ii) addition of a fat encapsulated microbial culture according to any one of items X1-X39 or a composition according to any one of items X40 or X41 to the yoghurt, and
   iii) post-pasteurization of the yoghurt of step ii).
W2. The method according to item W1, wherein the yoghurt of step i) has a pH in the range of 4.3-4.5.
W3. The method according to any one of items W1 or W2, wherein the yoghurt of step i) is a pasteurized yoghurt.
W4. The method according to any one of items W1-W3, wherein the post-pasteurization of step iii) is performed at a temperature of at least 60° C.
W5. The method according to any one of items W1-W4, wherein the post-pasteurization of step iii) is performed for at least 20 min, such as for 30 min.
W6. The method according to any one of items W1-W4, wherein the post-pasteurization of step iii) is performed at a temperature in the range of 65-75° C. for time period in the range of 2-10 minutes.
W7. The method according to any one of items W1-W4 or W6, wherein the post-pasteurization of step iii) is performed at a temperature of 70° C. for 5 minutes.
W8. The method according to any one of items W1-W7, wherein the content of microbial culture in the PPY is in the range of 1.0E+05 to 5.0E+09 CFU/g, preferably in the range of 1.0E+06 to 5.0E+08 CFU/g.
Z1. A fat encapsulated microbial culture according to any one of items X1-X39 or a composition according to any one of items X40 or X41 obtainable by a method according to any one of items Y1-Y8.
Z2. A product comprising a fat encapsulated microbial culture or a composition according to item Z1, wherein said product is selected from the group consisting of a feed, a plant health product, a food, a beverage and a pharmaceutical product.
Z3. A dairy product comprising a fat encapsulated microbial culture or a composition according to item Z1, wherein said dairy product is selected from the group consisting of yoghurt, cheese, butter, an inoculated sweet milk and a liquid fermented milk product.
Z4. The dairy product according to item Z3, wherein said dairy product is a post-pasteurized yoghurt (PPY).
Q1. Use of a fat encapsulated microbial culture according to any one of items X1-X39 and Z1 or a composition according to any one of items X40, X41 or Z1 in a product selected from the group consisting of a feed, a plant health product, a food, a beverage and/or a pharmaceutical product.
Q2. Use of a fat encapsulated microbial culture according to any one of items X1-X39 and Z1 or a composition according to any one of items X40, X41 or Z1 in a dairy product selected from the group consisting of yoghurt, cheese, butter, an inoculated sweet milk and a liquid fermented milk product.
Q3. The use according to item Q2, wherein said dairy product is a post-pasteurized yoghurt (PPY).

The invention will now be described in further details in the following non-limiting examples.

### EXAMPLES

### Example 1: Preparation of Freeze-Dried Probiotic Formulation and Measurement of CFU/g

Method:

Lacticaseibacillus rhamnosus GG (LGG, ATCC 53103) was fermented at a temperature of 40° C. and pH 5.5. The resulting fermentation broth was centrifuged followed by addition of cryoprotectants to improve survivability of the cells during freeze-drying process. The cryoprotectants consisted of sodium ascorbate, sucrose and maltodextrin DE12.

Samples were frozen to pellets immediately in liquid nitrogen (−196° C.) and the pre-freeze-dried material (PFD) was stored at −50° C. until freeze-drying. The freeze-drying took place in a Hetosicc freeze dryer at 32° C. and 0.3 mBar. Freeze-dried material (FD) was stored at −50° C. until further analysis.

Survivability and stability of LGG upon freezing and freeze-drying (FD) processes were evaluated by determination of the number of cells that were able to grow and divide before and after freezing and FD.

Each FD preparation were diluted 100-fold in a stomacher bag and then resuspended in sterile peptone water. Mixing was performed in Stomacher for 2×120 seconds at normal speed. The samples were then further diluted by transferring 1 mL into dilucups with 9 mL peptone water, and each dilution was mixed in a dilushaker, before preparing the next dilution.

1 mL of the appropriate dilution was transferred into a petri dish and 46° C. warm MRS (pH 6.5) agar was poured for the pour plating. The plate was mixed thoroughly by rotating in both clockwise and anti-clockwise direction, to ensure a homogenous dilution. Each dilution was performed in duplicates, and the plates were incubated at 37° C. for 3 days under anaerobic conditions, using Anaerogen (Oxoid, Thermo Scientific).

After incubation, the colonies were counted manually, and the results were expressed as CFU/g.

Results:

The freeze-dried LGG formulation obtained as described above resulted in formulations having a viability of 6.4E+11 CFU/g.

Conclusion:

The present example demonstrates that freeze-dried (FD) LGG can be prepared with minimal loss of viability.

### Example 2: Fat-Matrix Encapsulation of Freeze-Dried Powders of Probiotics

Method:

Prior to encapsulation, 10 g of FD-pellets were ground in the coffee grinder for 3×10 seconds, while allowing 10 seconds rest in between the grinding steps, to avoid the extra heat produced by the coffee grinder. Immediately after grinding, FD-powder was sealed in the aluminium bags and stored at −55±5° C. The FD powder was taken out from −55±5° C. and kept at room temperature for 30 minutes before encapsulation.

For encapsulation, two types of fat with high melting temperature (Akosoft 36 and Akofine R, both from AAK) and one emulsifier (Polyglycerol polyricinoleate (PGPR), Plasgaard) were utilised. 130 g of Akosoft 36, 300 g of Akofine R, and 20 g PGPR were weighed. The fat blend was mixed at 75° C. by continuous stirring with a ruston type turbine with 10 angular paddles attached to an IKA Eurostar power control visc, until all the ingredients were entirely solubilized as a molten fat blend. 10 g of FD powder (kept at 22±5° C. for 30 minutes) was added in 90 g molten fat blend at approx. 75±3° C. and stirred for 10-20 seconds with an IKA T25 digital ultra turrax.

After mixing, the suspension was pelletized in liquid nitrogen and stored at −55±5° C. Microencapsulated pellets were again grinded in a coffee grinder for 10 seconds, and microencapsulated freeze-dried powder (ME-FD powder) was stored at −55±5° C. until further experiments.

An exemplary, and non-limiting, method for preparation of fat encapsulated FD probiotics may be summarised as below:

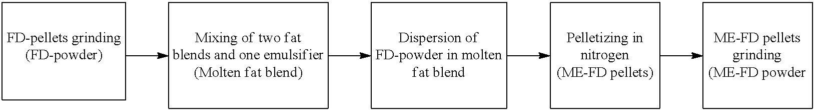

Results:

The fat encapsulated FD LGG showed a viability of 6.3E+10 CFU/g subsequent to microencapsulation compared to the non-encapsulated freeze-dried LGG formulation as described in Example 1 having a viability of 6.4E+11 CFU/g. This decrease in CFU/g of approximately an order of magnitude is expected as the addition of the fat coating dilute the concentration of LGG 10-fold.

Conclusion:

Thus, from the present example it can be concluded that fat encapsulation of freeze-dried LGG does not cause a loss in viable microorganisms (method variation of ±0.3 CFU/g). Accordingly, a significant pool of viable microorganisms is available for further post-processing of the fat encapsulated freeze-dried microorganisms.

### Example 3: Survivability of Free or Fat Encapsulated FD Probiotics Upon Batch Pasteurization

Method:

To evaluate the survivability of the free (not encapsulated) and fat encapsulated FD LGG preparations in a model yogurt at pH 4.5 upon batch pasteurization, cell counts were done before heating and after heating at 63° C. for 30 minutes in a skimmed milk matrix at pH 4.5.

Briefly, the pH of the skimmed milk was dropped to 4.5 by adding 2% of Glucone Delta Lactone (GDL) SG-E575 (Roquette) at 5° C., overnight. The next day, 40 g of acidified milk was weighed in 50 mL sterile tubes and 5.0E+07 CFU/g of either free or fat encapsulated culture was added. Free FD-pellets were mixed by continuous hand mixing for 10 seconds. Fat encapsulated FD powder samples were first mixed by hand stirring with a sterile spatula and then vortexed for 30 seconds at maximum speed. Afterward, these samples were mixed on a blood rotator at 5° C. for 30 minutes at 40 rpm.

After mixing both free FD-pellets and fat encapsulated FD powder samples were heat-treated at 63° C. for 30 minutes in a water bath. After heat treatment, samples were homogenized by stomacher for 120 seconds, at normal speed.

Cell counts were done on the samples both before and after the heat treatment.

For free (not encapsulated) LGG FD preparations, cell count was performed as follows. 10-fold of the first dilution was made into dilucups with 9 mL sterile peptone water. The further dilutions were made in the same way by transferring 1 mL into next dilucups with 9 mL peptone water. Each dilution was mixed in a dilushaker before preparing the next dilution, and after that, 1 mL of the appropriate dilution was transferred into the petri dish. The pour plating was done by adding 46° C. warm MRS (pH 6.5) agar. The plate was mixed thoroughly by rotating in both clockwise and anti-clockwise direction, to ensure a homogenous dilution. The plates were incubated at 37° C. for 3 days under anaerobic conditions, using Anaerogen. After incubation, the colonies counted manually, and the results expressed as CFU/g.

For fat encapsulated LGG FD preparations, cell count was performed using sterile peptone water with tween 80 at 40° C. Briefly, 10-fold of the first dilution was made into dilucups with 9 mL sterile peptone water with 1% Tween 80 (Merck) at 40° C. 100-fold of the first dilution was made in a stomacher bag by weighing 1 g of fat encapsulated FD powder to 99 g of sterile peptone water followed by mixing for 2×120 seconds at normal speed. Then, the sample was incubated at 40° C. for 30 minutes and mixed once again for 120 seconds at normal speed. Finally, the dilution series, pour plating, incubation and CFU/g determination were performed as described for the free LGG FD preparations above. Tween 80 and Polysorbate 80 is used herein interchangeably.

Results:

The results of cell survival in log₁₀ loss (CFU/g) upon post-pasteurization are shown in Table 2. Free (not encapsulated) FD LGG and fat encapsulated FD LGG both showed good survivability by having 1.99 and 2.03 log₁₀ loss (CFU/g), respectively.

| | Before post- | | After post- | | Log₁₀ Loss |
|---|---|---|---|---|---|
| | pasteurization | | pasteurization | | (CFU/g) |
| Sample | CFU/g | STDEV | CFU/g | STDEV | After Heating |
| LGG | 4.85E+06 | 4.95E+05 | 4.95E+04 | 7.78E+03 | 1.99 |
| ME - | 3.20E+07 | 1.41E+06 | 2.98E+05 | 5.94E+04 | 2.03 |
| LGG | | | | | |

Table 2. Shows survivability of cell (cell count in CFU/g) for freeze-dried samples of free LGG (“LGG”) and fat encapsulated LGG (“ME-LGG”) after batch pasteurization at 63° C. for 30 minutes in a skimmed milk matrix at pH 4.5.

Conclusion:

The present example demonstrates that freeze-dried LGG in both free and fat encapsulated form can survive the post-pasteurisation step.

### Example 4: Effect of Fat Encapsulation on Survivability and Post-Acidification of Cryo Formulated Probiotics During Storage

Method:

The stability and post-acidification of both free FD LGG-pellets and fat encapsulated freeze-dried LGG powder in model yoghurt at pH 4.5 was measured upon extended storage.

After doing batch pasteurization as described in Example 3, the samples were kept at 5° C. or 25° C. to determine the shelf life of free and fat encapsulated LGG during 8 weeks of storage. The effect of temperature and post-acidification on the shelf life of the cultures were measured at five-time points (T_{1day}, T_{14days}, T_{28days}, T_{42days}, and T_{56days}) by doing cell count and measuring pH. For the cell counts, these samples were mixed on a blood rotator at 5° C. for 30 minutes at 40 rpm. Cell counts were determined as described in Example 3.

Results:

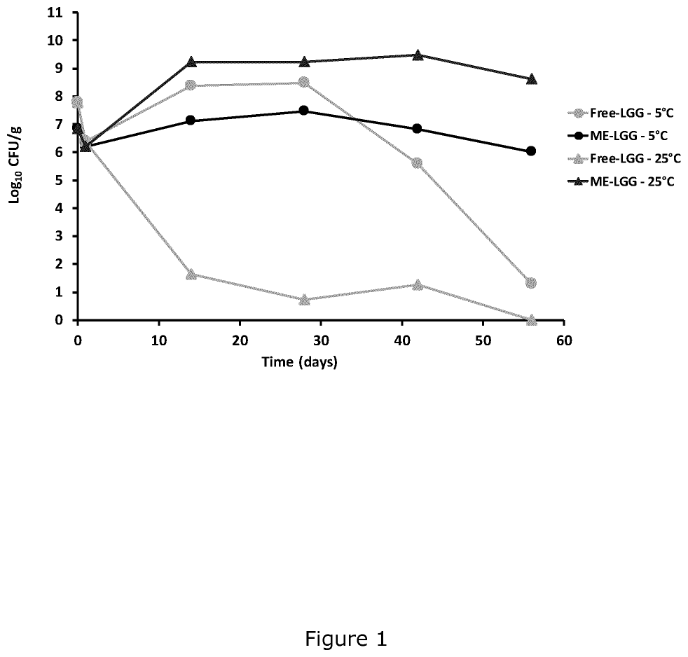

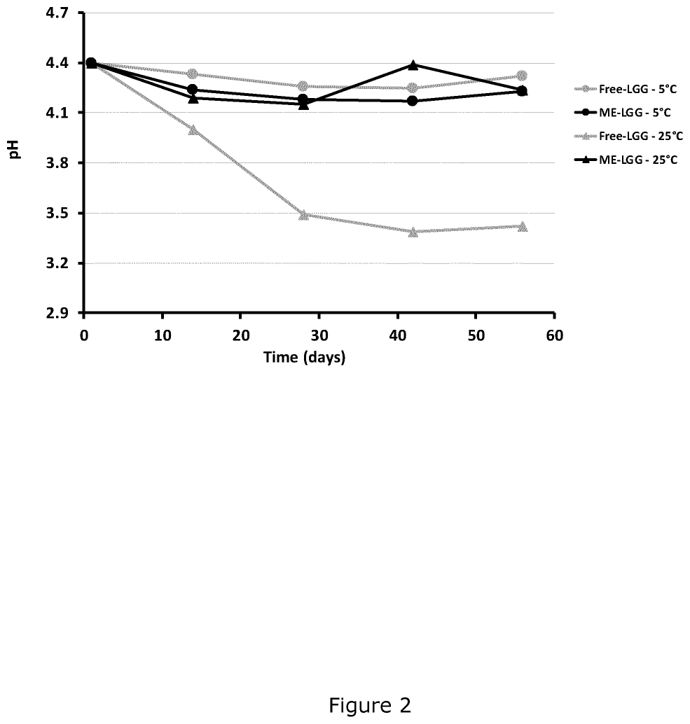

The blank corrected cell counts of survival cells for both Free-LGG and ME-LGG in post pasteurized model yogurt during storage at 5° C. and 25° C. for 56 days is shown in FIG. 1. The result shows a significant loss of cell numbers in the free FD LGG sample stored at both 5° C. and 25° C. over a period of 56 days. At the same time, a decrease in pH was noticed in the free FD LGG sample at 25° C. (FIG. 2), which indicates post-acidification over storage. In contrast, fat encapsulated FD LGG retained viability with low post-acidification when stored at 25° C. for 56 days.

Conclusion:

This example demonstrates that fat encapsulation efficiently enhances the ability of probiotics to retain viability and resist post-acidification upon storage for prolonged periods of time at ambient temperatures.

### REFERENCES

Seifert & Mogensen (2002), Bulletin of the IDF, 377, 10-19
Zheng et al. (2020), Int. J. Syst. Evol. Microbiol., 70, 2782-2858

1. A fat encapsulated microbial culture comprising:
i) a preparation comprising a microbial culture, and
ii) an encapsulation matrix comprising one or more fat components, each of which fat components having a melting point of at least 25° C.

2. The fat encapsulated microbial culture according to claim 1, wherein the one or more fat components have a melting point of at least 30° C., such as at least 31° C., such as at least 32° C., such as at least 33° C.

3. The fat encapsulated microbial culture according to any one of the preceding claims, wherein the one or more fat components are selected from the group consisting of butterfat, cocoa butter, coconut fat, shea butter, mango kernel fat, palm oil, palm kernel oil, lard, hydrogenated oils, interesterified oils, and combinations thereof.

4. The fat encapsulated microbial culture according to any one of the preceding claims, wherein the encapsulation matrix further comprises at least one emulsifier.

5. The fat encapsulated microbial culture according to any one of the preceding claims, wherein the preparation is a dry preparation.

6. The fat encapsulated microbial culture according to any one of the preceding claims, wherein the microbial culture is a probiotic culture.

7. The fat encapsulated microbial culture according to claim 6, wherein the probiotic culture is of a genus selected from the group consisting of Lactobacillus or Bifidobacterium.

8. The fat encapsulated microbial culture according to any one of the preceding claims, wherein the microbial culture is a lactic acid bacteria (LAB).

9. A composition comprising the fat encapsulated microbial culture according to any one of the preceding claims.

10. A dairy product comprising a fat encapsulated microbial culture according to any one of claims 1-8 or a composition according to claim 9.

11. The dairy product according to claim 10, wherein said dairy product is a post-pasteurized yoghurt (PPY).

12. A method for preparing a fat encapsulated microbial culture according to any one of claims 1-8 or a composition according to claim 9, said method comprising the steps of:
i) provision of a preparation comprising a microbial culture,
ii) provision of an encapsulation matrix, and
iii) mixing the encapsulation matrix with the preparation to form a microencapsulated microbial culture,
wherein the encapsulation matrix comprises one or more fat components, each of which fat components having a melting point of at least 25° C.

13. A fat encapsulated microbial culture according to any one of claims 1-8 or a composition according to claim 9 obtainable by a method according to claim 12.

14. Use of a fat encapsulated microbial culture according to any one of claim 1-8 or 13 or a composition according to any one of claim 9 or 13 in a product selected from the group consisting of a feed, a plant health product, a food, a beverage and/or a pharmaceutical product.

15. A method for preparing a post-pasteurized yoghurt (PPY), said method comprising the steps of:
i) provision of a yoghurt with a pH in the range of 4.0-4.6,
ii) addition of a fat encapsulated microbial culture according to any one of claims 1-8 or a composition according to claim 9 to the yoghurt, and
iii) post-pasteurization of the yoghurt of step ii).
